(19) United States
(12) Patent Application Publication
(10) Pub. No.: US 2015/0260729 A1
(43) Pub. Date: Sep. 17, 2015

(54) SCHIZOPHRENIA MARKER SET AND ITS UTILIZATION

(71) Applicant: NATIONAL UNIVERSITY CORPORATION NAGOYA UNIVERSITY, Aichi (JP)

(72) Inventors: Norio Ozaki, Nagoya-shi (JP); Taku Nagai, Nagoya-shi (JP); Akira Yoshimi, Nagoya-shi (JP); Shinnosuke Yamada, Nagoya-shi (JP); Akihiro Hirakawa, Nagoya-shi (JP); Shohko Kunimoto, Nagoya-shi (JP); Yurie Matsumoto, Nagoya-shi (JP)

(21) Appl. No.: 14/434,207

(22) PCT Filed: Oct. 3, 2013
(86) PCT No.: PCT/JP2013/076918

§ 371 Date: Apr. 8, 2015

(30) Foreign Application Priority Data
Oct. 15, 2012 (JP) 2012-228417

(51) Int. Cl. G01N 33/68

(52) U.S. Cl. CPC G01N 33/6893; G01N 2800/302; G01N 2800/50

(57) ABSTRACT

The object is to provide a combination of biomarkers which allow highly accurate discrimination of schizophrenia, and a utilization thereof. A combination of schizophrenia marker set including a combination of two or more protein molecules selected from the group consisting of trifunctional purine biosynthetic protein adenosine-3, uroporphyrinogen decarboxylase, interferon-induced GTP-binding protein Mx1, glutaredoxin-3, microtubule-associated protein RP/EB family member 1, tubulin folding cofactor B, immunoglobulin mu chain C region, and heat shock 70 kDa protein 4L is provided. In addition, a method for examining schizophrenia using the level of the marker set in a specimen as an index is provided.

### TECHNICAL FIELD

The present invention relates to a schizophrenia marker useful for the examination of schizophrenia, and more specifically to a combination of biomarkers (biomarker set) usable for the determination of onset possibility (prevalence rate) of schizophrenia, and a method for examining schizophrenia using the same. The present application claims priority based on Japanese Patent Application No. 2012-228417 filed on Oct. 15, 2012, and the content of the patent application is hereby incorporated by reference in its entirety.

### BACKGROUND ART

Schizophrenia is a chronic and progressive mental disorder developed during puberty and adolescence, and its main symptoms are positive symptoms (hallucinations and delusion, disorganized speech/behavior), negative symptoms (for example, flat affect, poverty of thought content, and loss of motivation) and cognition disorder (attentional dysfunction, poor working memory, and executive function deficits). Schizophrenia is regarded as a multifactorial disorder involved with genetic and environmental factors, and there are still many unknown points regarding the molecular biological mechanism of the disease state of schizophrenia, and thus no biochemical test method has been found.

Diagnosis of schizophrenia is carried out by diganostics (symptomatology) based on medical health questionnaire and patient's complaint, and there is no established diagnosis method by more empirical methods such as biochemical examination or similar. Schizophrenia progresses in several years after development, irreversible damage is caused, which likely causes permanent disturbance in social function. However, therapeutic intervention is delayed because there is no examination method useful for diagnosis, so that considerable cases are advancing in severity. Development of an examination method useful for the diagnosis of schizophrenia is desired. However, this disease is regarded as developed by disorders of cerebral molecules, while it is very difficult to detect the molecular pathogenesis in the brain, so that the development of the examination method is not proceeding.

From the result of studies such as twin studies, it was proved that hereditary factors are markedly involved with the development of schizophrenia. Some candidate genes which are suggested to be involved with the development of schizophrenia have been identified by gene analysis using the patients with schizophrenia and healthy subjects (for example, Non-Patent Literatures 1 to 3), but there are few clues for clarifying the association between these genes and the disease state of schizophrenia, so that the molecular mechanism developing the disease has not been elucidated. As a result of this, pathophysiology of the disease is still unknown, so that there is no established diagnosis method based on biochemical examination.

### CITATION LIST

### Patent Document

Patent Literature 1: Japanese Unexamined Patent Application Publication No. 2012-013415

### Non-Patent Document

Non-Patent Literature 1: Arcos-Burgos M. et al., Am J Hum Genet 77,6 p 937-44, 2005
Non-Patent Literature 2: Lewis C M. et al., Am J Hum Genet 73, 34-48, 2003
Non-Patent Literature 3: Badner J A. And Gershon E S., Mol Psychiatry 7, 405-11, 2002

### SUMMARY OF THE INVENTION

### Problem of be Solved by Invention

The present invention is intended to provide a combination of biomarkers and utilization thereof (for example, an examination method, an examination reagent, and an examination kit) which allow highly accurate discrimination of schizophrenia.

### Means for Solving Problem

In order to find biomarkers specific to schizophrenia, the inventors carried out researches with focus on the lymphocytes contained in the peripheral blood. As a result of this, 22 proteins which showed a significant difference between the patient and healthy subject groups in the expression level were reported (Patent Literature 1). Most of the identified proteins were found to be expressed in the brain. Therefore, the possibility of their deep involvement with pathogenesis of schizophrenia was strongly suggested.

Each of the 22 identified proteins is highly useful. However, high discrimination accuracy as possible is demanded in clinical fields, and such demand is hard to be satisfied by a single biomarker. In addition, in order to achieve further higher accuracy in the discrimination of schizophrenia or other disease developed by various factors, combination of a plurality of biomarkers is considered advantageous. From this viewpoint, the identification of the combinations of biomarkers which allow highly accurate discrimination was aimed using a statistical method. As a result of this, markedly effective biomarkers were found, and the combinations of biomarkers that bring about very high discrimination accuracy were successfully identified. The examination using the combination of the specified biomarkers is markedly practical, and is expected to contribute to dramatic improvement in diagnosis techniques of schizophrenia. The present invention described below is mainly based on the above results.

[1] A schizophrenia marker set including a combination of two or more protein molecules selected from the group consisting of trifunctional purine biosynthetic protein adenosine-3, uroporphyrinogen decarboxylase, interferon-induced GTP-binding protein Mx1, glutaredoxin-3, microtubule-associated protein RP/EB family member 1, tubulin folding cofactor B, immunoglobulin mu chain C region, and heat shock 70 kDa protein 4L.

[2] The schizophrenia marker set of [1], which includes at least trifunctional purine biosynthetic protein adenosine-3, uroporphyrinogen decarboxylase, interferon-induced GTP-binding protein Mx1, and glutaredoxin-3.

[3] The schizophrenia marker set of [2], which further includes microtubule-associated protein RP/EB family member 1 and/or tubulin folding cofactor B.

[4] The schizophrenia marker set of [1], which is a combination of trifunctional purine biosynthetic protein adenosine-3, uroporphyrinogen decarboxylase, interferon-induced GTP-binding protein Mx1, glutaredoxin-3, microtubule-associated protein RP/EB family member 1, and tubulin folding cofactor B.

[5] The schizophrenia marker set of [1], which is a combination of trifunctional purine biosynthetic protein adenosine-3, uroporphyrinogen decarboxylase, interferon-induced GTP-binding protein Mx1, and glutaredoxin-3.

[6] The schizophrenia marker set of [1], which includes at least trifunctional purine biosynthetic protein adenosine-3 and interferon-induced GTP-binding protein Mx1.

[7] The schizophrenia marker set of [1], which is a combination of trifunctional purine biosynthetic protein adenosine-3 and interferon-induced GTP-binding protein Mx1.

[8] The schizophrenia marker set of [1], which includes one or more protein molecules selected from the group consisting of trifunctional purine biosynthetic protein adenosine-3, interferon-induced GTP-binding protein Mx1, and heat shock 70 kDa protein 4L.

[9] An examination method for schizophrenia using the level of the schizophrenia marker set of any one of [1] to [8] in the specimen as an indication.

[10] The examination method for schizophrenia of [9], which includes the following steps (1) to (3):

(1) a step of preparing a specimen derived from the subject;

(2) a step of detecting the protein molecules composing the marker set in the specimen; and

(3) a step of determining the present or future development possibility of schizophrenia based on the detection result.

[11] The examination method for schizophrenia of [10], wherein a lower detected value shows a higher development possibility of schizophrenia for trifunctional purine biosynthetic protein adenosine-3, uroporphyrinogen decarboxylase, glutaredoxin-3, microtubule-associated protein RP/EB family member 1, tubulin folding cofactor B, and heat shock 70 kDa protein 4L, and a higher detected value shows a higher development possibility of schizophrenia for interferon-induced GTP-binding protein Mx1 and immunoglobulin mu chain C region.

[12] The examination method for schizophrenia of [10] or [11], wherein the determination of step (3) is carried out using a logistic model.

[13] The examination method for schizophrenia of any one of [9] to [12], wherein the specimen is blood, blood plasma, blood serum, oral mucosa, nasal mucosa, skin, blood cells, or lymphoblastoid cells prepared by immortalizing the blood lymphocytes collected from the subject.

[14] A kit for examining schizophrenia including a reagent for detecting the protein molecules composing the schizophrenia marker set of [1], and an instruction manual.

[15] The kit for examining schizophrenia of [14], wherein each of the reagents is an antibody to the target protein molecule.

### BRIEF DESCRIPTION OF THE DRAWINGS

FIG. 1 shows the result of expression analysis (Western blot test and 2D-DIGE method). The S/C (the ratio between the schizophrenia patient group and the healthy subject group) and the p value (Student's t-test) of each analysis are shown.

FIG. 2 shows the characteristics of the top 30 models with a higher corrected AUC.

FIG. 3 shows the odds ratio of protein contained in the candidate model 1.

FIG. 4 shows the odds ratio of protein contained in the candidate model 2.

FIG. 5 shows the validation of effectiveness of the selected biomarkers.

The 30 specimens used for screening the biomarkers (screening data) and other 30 specimens (validation data) were used for the validation.

FIG. 6 shows the validation of the effectiveness of the selected biomarkers. The validation results are summarized together with the explanation of the functions of the biomarkers.

FIG. 7 shows the validation of the four biomarkers (MX1, GART, UROD, and GLRX3) using a validation data set. The evaluation used a four-variable model.

FIG. 8 shows the validation of the four biomarkers (MX1, GART, UROD, and GLRX3) using a validation data set. The predicting performance of the four-variable model for the validation data was evaluated. The AUC was 0.724. In consideration of the number of data (N=30), reproducibility is high.

FIG. 9 shows the validation of the six biomarkers (MX1, GART, UROD, GLRX3, MAPRE1, and TBCB) using the validation data set. The evaluation used a six-variable model.

FIG. 10 shows the validation of the six biomarkers (MX1, GART, UROD, GLRX3, MAPRE1, and TBCB) using a validation data set. Prediction performance of the six-variable model for the validation data was evaluated. The AUC was 0.664.

FIG. 11 shows the correlation between MX1, GART, and HSPA4L (correlation coefficient for the validation data, and the correlation coefficient integrating the both data).

FIG. 12 shows various predicting models and their discrimination accuracies. The upper four predicting models constructed the predicting models based on screening data sets, and evaluated them based on a validation data set. The middle four predicting models constructed the predicting models based on validation data sets, and evaluated them based on screening data sets. The lower four predicting models constructed the predicting models based on screening and validation data sets. S: screening data set, V: validation data set, I: integration of screening and validation data sets.

### DESCRIPTION OF EMBODIMENTS

### 1. Schizophrenia Marker Set

A first aspect of the present invention relates to a schizophrenia marker set including a combination of protein molecules (biomarkers) specific to schizophrenia (hereinafter may be referred to as “the biomarker set of the present invention”). The biomarker set of the present invention is useful for the determination and evaluation of present or future development possibility of schizophrenia. The “present development possibility” represents whether the patient is affected by or has developed schizophrenia (whether the patient has schizophrenia or not) at the time of inspection, or the probability of onset or development. On the other hand, the “future development possibility” represents future possibility (risk) of development of schizophrenia. “Schizophrenia” is a generic name of psychiatric disorders based on fragmentation of mental functions. According to the International Statistical Classification of Diseases and Related Health Problems (ICD) by the World Health Organization (WHO), the schizophrenia is classified into Paranoid schizophrenia, Hebephrenic schizophrenia, Catatonic schizophrenia, Undifferentiated schizophrenia, Post-schizophrenic depression, Residual schizophrenia, and Simple schizophrenia.

The biomarker set of the present invention is a combination of protein molecules that were found to be correlated with schizophrenia. More specifically, the biomarker set of the present invention is composed of two or more protein molecules selected from the eight proteins listed below. The biomarker set of the present invention is composed of preferably three or more, more preferably four or more protein molecules:

trifunctional purine biosynthetic protein adenosine-3 (hereinafter referred to as “GART”, SEQ ID NO. 1);

uroporphyrinogen decarboxylase (hereinafter referred to as “UROD”, SEQ ID NO. 2);

interferon-induced GTP-binding protein Mx1 (hereinafter referred to as “MX1”, SEQ ID NO. 3);

glutaredoxin-3 (hereinafter referred to as “GLRX3”, SEQ ID NO. 4);

microtubule-associated protein RP/EB family member 1 (hereinafter referred to as “MAPRE1”, SEQ ID NO. 5);

tubulin folding cofactor B (hereinafter referred to as “TBCB”, SEQ ID NO. 6);

immunoglobulin mu chain C region (hereinafter referred to as “IGHM”, SEQ ID NO. 7); and

heat shock 70 kDa protein 4L (hereinafter referred to as “HSPA4L”, SEQ ID NO. 8).

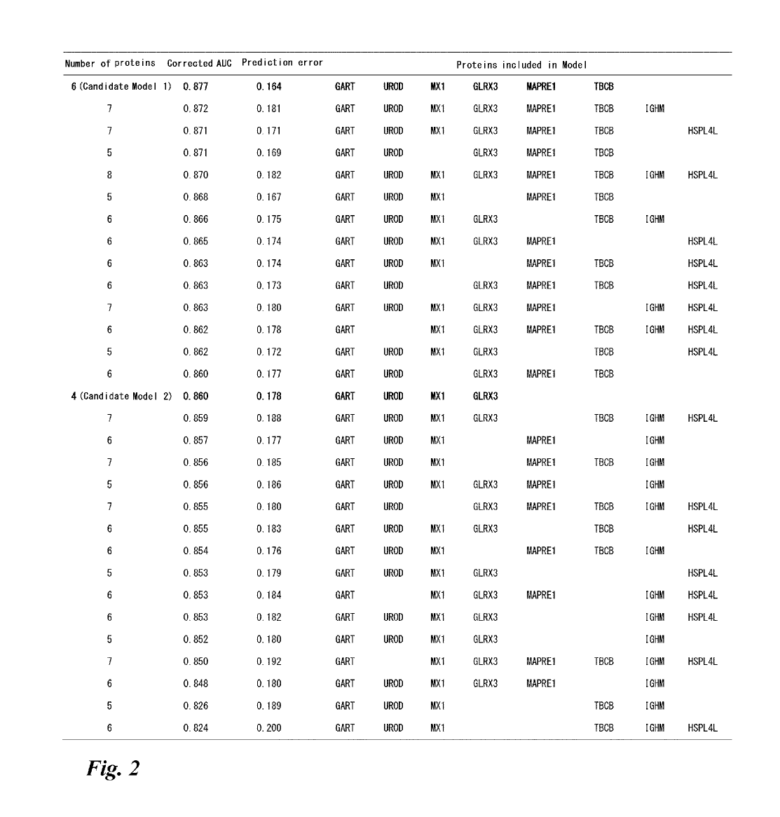

The biomarker set of the present invention may include various combinations of the above-described proteins. As shown in the below-described examples (in particular, the table of FIG. 2), GART, UROD, MX1, and GLRX3 are particularly useful and important for allowing highly accurate discrimination. Therefore, according to a preferred embodiment of the present invention, a schizophrenia marker set including at least the four protein molecules is provided. It was also shown that MAPRE1 and TBCB are useful secondary to the above-described four protein molecules. Therefore, it is also preferred that the above-described four protein molecules be combined with one or both of them to make a schizophrenia marker set.

Specific examples of the biomarker set of the present invention are listed below. They were specified as the combinations achieving high discrimination accuracy, as a result of the analysis by a statistical method:

a combination of GART, UROD, MX1, GLRX3, MAPRE1, and TBCB;

a combination of GART, UROD, MX1, GLRX3, MAPRE1, TBCB, and IGHM;

a combination of GART, UROD, MX1, GLRX3, MAPRE1, TBCB, and HSPA4L;

a combination of GART, UROD, GLRX3, MAPRE1, and TBCB;

a combination of GART, UROD, MX1, GLRX3, MAPRE1, TBCB, IGHM, and HSPA4L;

a combination of GART, UROD, MX1, MAPRE1, and TBCB;

a combination of GART, UROD, MX1, GLRX3, TBCB, and IGHM;

a combination of GART, UROD, MX1, GLRX3, MAPRE1, and HSPA4L;

a combination of GART, UROD, MX1, MAPRE1, TBCB, and HSPA4L;

a combination of GART, UROD, GLRX3, MAPRE1, TBCB, and HSPA4L;

a combination of GART, UROD, MX1, GLRX3, MAPRE1, IGHM, and HSPA4L;

a combination of GART, MX1, GLRX3, MAPRE1, TBCB, IGHM, and HSPA4L;

a combination of GART, UROD, MX1, GLRX3, TBCB, and HSPA4L;

a combination of GART, UROD, GLRX3, MAPRE1, and TBCB;

a combination of GART, UROD, MX1, and GLRX3;

a combination of GART, UROD, MX1, GLRX3, TBCB, IGHM, HSPA4L;

a combination of GART, UROD, MX1, MAPRE1, and IGHM;

a combination of GART, UROD, MX1, MAPRE1, TBCB, and IGHM;

a combination of GART, UROD, MX1, GLRX3, MAPRE1, and IGHM;

a combination of GART, UROD, GLRX3, MAPRE1, TBCB, IGHM, and HSPA4L;

a combination of GART, UROD, MX1, GLRX3, TBCB, and HSPA4L;

a combination of GART, UROD, MX1, MAPRE1, TBCB, and IGHM;

a combination of GART, UROD, MX1, GLRX3, and HSPA4L;

a combination of GART, MX1, GLRX3, MAPRE1, IGHM, and HSPA4L;

a combination of GART, UROD, MX1, GLRX3, IGHM, and HSPA4L;

a combination of GART, UROD, MX1, GLRX3, and IGHM;

a combination of GART, MX1, GLRX3, MAPRE1, TBCB, IGHM, and HSPA4L;

a combination of GART, UROD, MX1, GLRX3, MAPRE1, and IGHM;

a combination of GART, UROD, MX1, TBCB, and IGHM; and

a combination of GART, UROD, MX1, TBCB, IGHM, and HSPA4L.

Among the above combinations, the combinations of GART, UROD, MX1, GLRX3, MAPRE1, and TBCB showed the highest discrimination accuracy. In consideration of this fact, this combination is regarded as one of the optimum biomarker sets. In addition, according to the Occam's razor, which is a principle in statistics, the number of proteins used for discrimination is preferably smaller when the discrimination accuracy is at the same level (details will be described later). The combination of GART, UROD, MX1, and GLRX3 most conforms to this principle.

As shown in the below-described examples, as a result of validation of reproducibility, it was found that GART and MX1 have good reproducibility. In view of this finding, it is preferred that the biomarker set includes GART and MX1. In particular, it is preferred that the biomarker set be composed of GART and MX1. On the other hand, in consideration of the finding that GART and MX1 have good reproducibility and the finding that HSPA4L also has relatively good reproducibility, according to a preferred embodiment of the present invention, the biomarker set includes one or more protein molecules selected from the group consisting of GART, MX1, and HSPA4L.

### 2. Method for Examining Schizophrenia

A second aspect of the present invention relates to the utilization of the biomarker set of the present invention, and provides a method for examining the present or future development possibility of schizophrenia (hereinafter may be referred to as “the examination method of the present invention”). The examination method of the present invention is useful as a means for determining whether the patient is affected by or has developed schizophrenia, or a means for determining the possibility of future development of schizophrenia. The examination method of the present invention provides information useful for diagnosing schizophrenia. The examination method of the present invention allows simple and objective determination of development possibility of schizophrenia.

The delay of time from the development of a mental disease such as schizophrenia to the initiation of drug therapy is referred to as duration of untreated psychosis (DUP). It is known that the longer the DUP, the worse the clinical outcome. On the other hand, several years from the development are the important period, which influences the prognosis, and is referred to as critical period. According to a finding of cerebral nerve image analysis, it is reported that the longer the DUP, the smaller the volume of the planum temporale, which is a part of left gyrus temporalis superior (Takahashi et al.: Psychiatry Res 154: 209-219, 2007). This finding suggests that the changes in brain morphology proceeds during the untreated duration in the early stage (critical period) of the disease, and that it is very important to shorten the DUP, as well as to early intervene to improve the neurobiological changes in the critical period. The examination method of the present invention can be used also for screening of schizophrenia, and is useful for early detection and early treatment of schizophrenia.

The examination method of the present invention uses the level of the biomarker set of the present invention in the specimen derived from the subject as an indication. The “level” herein typically means the “amount” or “concentration”. However, according to the convention and technical common knowledge, the term “level” is used also for expressing whether the molecules to be detected can be detected or not (more specifically, the presence or absence of apparent presence). The biomarker set to be used may be selected in consideration of the demanded accuracy and convenience of the examination.

The examination method of the present invention carries out the following steps:

(1) a step of preparing a specimen derived from the subject;

(2) a step of detecting the protein molecules composing the marker set (more specifically, a combination of two or more (preferably three or more, more preferably four or more) protein molecules selected from the group consisting of GART, UROD, MX1, GLRX3, MAPRE1, TBCB, IGHM, and HSPA4L) in the specimen; and

(3) a step of determining the present or future development possibility of schizophrenia based on the detection result.

In the step (1), a specimen derived from a subject is prepared. Examples of the specimen include the blood, blood plasma, blood serum, oral mucosa, nasal mucosa, and skin of the subject. Alternatively, the blood cells derived from the subject may be used as the specimen. Alternatively, the lymphocytes, B-cells, or T-cells separated from the blood cells may be used as the specimen. Alternatively, the cells after subcultivation may be used. On the other hand, lymphoblastoid cells prepared by immortalizing the blood lymphocytes collected from the subject may be used as the specimen. The “lymphoblastoid cells” are the cells similar to the cells before differentiation into mature lymphocytes, and can be prepared by immortalizing the lymphocytes separated from the blood by Epstein-Barr virus. The established lymphoblastoid cells show a protein expression profile, which more highly reflects the features and characteristics of the disease in comparison with fresh blood cells. Accordingly, the use of the lymphoblastoid cells as the specimen prevents masking of the intrinsic expression profile (more specifically, the expression profile to be detected by the method of the present invention) by the conditions of the subject (condition-dependent factors) at the time of collection of the cells, which are the elements other than the disease. As a result of this, an examination result with higher reliability which more highly reflects the features and characteristics of the disease can be obtained. The examination using, for example, the blood, blood plasma, or blood serum as the specimen is easy in operation, and thus is particularly useful as a screening examination. On the other hand, the examination using the lymphoblastoid cells as the specimen gives a result with higher reliability, and thus is particularly useful as a means for secondary examination or definite diagnosis. Accordingly, highly efficient and reliable diagnosis is achieved by targeting the high-risk group (the subjects with high development possibility) by the examination using, for example, the blood, blood plasma, or blood serum as the specimen, followed by carrying out the examination using the lymphoblastoid cells as the specimen.

The subject is not particularly limited. More specifically, the present invention is widely applicable to the subjects requiring the determination of the present or future development possibility of schizophrenia (more specifically, the presence or absence of onset or development of schizophrenia, the degree of possibility of development of schizophrenia, and the degree of possibility of future development of schizophrenia). For example, when the present invention is applied to a patient who has been diagnosed to have schizophrenia by medical health questionnaire, propriety of the diagnosis can be reviewed based on an objective index, or the level of protein expression. More specifically, the examination method of the present invention provides information, which helps or supports prior art diagnosis. This information is useful for the decision of more appropriate treatment policy, and promotes the improvements in therapeutic effect and patient's QOL (Quality of Life). On the other hand, the present invention can be used to monitor the disease condition, thereby preventing the increase in obstinacy and seriousness, and recurrence.

Those presumed to have a high risk of onset of schizophrenia based on the family background and other factors (high risk subjects) are also preferred subjects. The application of the present invention to the subject before development of the symptoms of schizophrenia allows inhibition or delay of the development, or therapeutic intervention in the early stage of the disease. The present invention is also useful for identification of those having a high risk of onset of schizophrenia. This identification allows, for example, the decrease in the development possibility (possibility of acquiring disease) owing to precautions and improvements in the lifestyle habit. Those who cannot or are difficult to be determined whether they have schizophrenia or not by prior art diagnosis, such as those having no subjective symptom, are also preferred subjects in the present invention. The present invention may be carried out as a term of health examination.

In the step (2), the biomarkers in the specimen are detected. It is not essential to strictly determine the level of each biomarker. More specifically, the level of the biomarkers is detected to a degree that allows the determination of development possibility of schizophrenia in the subsequent step (3). For example, the detection may be carried out so as to allow the discrimination whether the level of the biomarkers in the specimen exceeds the predetermined criterion value or not.

The method for detecting each biomarker is not particularly limited, but preferably an immunological method is used. An immunological method allows prompt and highly sensitive detection. In addition, operation is easy. Measurement by an immunological method uses a substance having specific binding property to the biomarker to be used. The substance is usually an antibody, but may be not an antibody as long as the substance has specific binding property to the biomarker, and the amount of binding is measurable. The antibody may be not a commercially available antibody, and may be newly prepared by, for example, an immunological method, a phage display method, or a ribosome display method.

Examples of the measurement method include latex agglutination assay, fluoroimmunoassay (FIA method), enzyme immunoassay (EIA method), radioimmunoassay (RIA method), and Western blot test method. Examples of the preferred measurement method include the FIA method and EIA method (including ELISA method). These methods allow high-sensitivity, prompt, and convenient detection. The FIA method uses a fluorescent-labeled antibody, and detects an antigen-antibody complex (immune complex) using fluorescence as a signal. On the other hand, the EIA method uses an enzymatically labeled antibody, and detects an immune complex using color development or luminescence based on enzyme reaction as a signal.

The ELISA method has many advantages such as high detection sensitivity, high specificity, marked quantitative determination performance, easy operation, and suitability for simultaneous processing of multiple specimens. An example of the specific operation method using the ELISA method is described below. Firstly, an anti-biomarker antibody is immobilized on an insoluble support. Specifically, for example, the surface of a microplate is immunized (coated) with an anti-biomarker monoclonal antibody. The specimen is brought into contact with the immobilized antibody. As a result of this operation, an immune complex is formed if the antigen (protein molecule as the biomarker) to the immobilized anti-biomarker antibody is present in the specimen. After the non-specific binding component is removed by washing operation, the immune complex is labeled by adding an enzyme-bound antibody, and then color development by reaction with the enzymatic substrate is carried out. Subsequently, the immune complex is detected using the amount of color development as the index. Details about the ELISA method are described in many publications and literatures, and establishment of the experimental procedure and conditions of the methods may refer to them. A competition method (a method including addition of an antigen and a specimen for competition) may be used in place of non-competition method. Alternatively, a method directly detecting the biomarker in the specimen with a labeling antibody, or a method for sandwich method may be used. The sandwich method uses two antibodies having different epitopes (trapping antibody and detecting antibody).

A means that allows simultaneous detection of multiple specimens, such as a protein array or protein chip, may be used. As the probe, for example, an antibody specific to the target biomarker is used.

In the step (3), present or future development possibility of schizophrenia is determined based on the detection result. In order to allow highly accurate determination, the determination is preferably carried out after comparing the detected value obtained in the step (2) with the detected value of the control specimen. The determination of the development possibility may be qualitative or quantitative. As is evident from the criteria for determination, the determination herein may be carried out automatically or mechanically without depending on diagnosis by specialists such as physicians or laboratory technician.

In the present invention, in principle, the following criteria are applied to each biomarker. More specifically, for GART, UROD, GLRX3, MAPRE1, TBCB, and HSPA4L, the development possibility of schizophrenia is high when the detected value is low (the detected value and development possibility are in negative correlation with each other), and for MX1 and IGHM, the development possibility of schizophrenia is high when the detected value is high (the detected value and development possibility are in positive correlation with each other).

The determination in the step (3) may be carried out using a logistic model (statistical model) corresponding to the biomarker set to be used. A specific example (the case using the biomarker set composed of GART, UROD, MX1, GLRX3, MAPRE1, and TBCB) is described below.

### <Determination Using Logistic Model>

In the following logistic model, the p is calculated. Specifically, the level (detected value) of each protein molecule is assigned to the following formula to obtain the development possibility of schizophrenia (prevalence rate of schizophrenia).

$$\begin{matrix}[ Formula 1 ] & \\ p = \frac{exp ( \begin{matrix}\begin{matrix}11.54 - 4.64 GART - 2.67 UROD + \\ 2.04 MX 1 - 3.14 GLRX 3 -\end{matrix} \\ 1.65 MARPRE 1 - 2.01 TBCB\end{matrix} )}{1 + exp ( \begin{matrix}\begin{matrix}11.54 - 4.64 GART - 2.67 UROD + \\ 2.04 MX 1 - 3.14 GLRX 3 -\end{matrix} \\ 1.65 MARPRE 1 - 2.01 TBCB\end{matrix} )} & \end{matrix}$$

Alternatively, a criterion value or cutoff value is established, and qualitative determination such as the following (a) or (b) may be made. The criterion value or cutoff value used for determination may be decided in consideration of the type and condition of the specimen used, and demanded accuracy (confidence level).

(a) The case where all the biomarkers contained in the biomarker set are positive (cutoff value or more) is determined as positive (for example, development possibility is 50% or more), and the other case is determined as negative (for example, development possibility is less than 50%).

(b) The case where at least one of the biomarkers contained in the biomarker set is positive (cutoff value or more) is determined as positive (for example, development possibility is 50% or more), and the other case is determined as negative (for example, development possibility is less than 50%).

Alternatively, a plurality of determination categories may be provided, and subjected to quantitative or semiquantitative determination. When quantitative determination is made, for example, a plurality of determination categories related to the ranges of the level of the biomarkers (development possibility of schizophrenia is defined for each determination category) are established in advance, and the determination category of the specimen is specified from the level of the biomarkers. The number of the determination categories, and the level of the biomarkers and determination result related to the determination categories are not particularly limited, and may be freely established based on, for example, a preliminary experiment. For example, the “threshold” determining the level of the development possibility using the predetermined threshold as the border, and “the range of biomarker level” relating to the category concerning with the level of development possibility can be decided by statistical analysis using many specimens. When the analysis is carried out using statistical processing, it is commonly effective to establish high risk and low risk groups. Examples of the high-risk group include the group of patients with schizophrenia and the group of those having many family members with schizophrenia, and examples of the low risk group include the group of healthy subjects and the group without family member with schizophrenia.

According to one embodiment of the present invention, in one subject, the biomarker level measured at one point is compared with the biomarker level measured past, and the presence or absence and/or the degree of increase and decrease of each biomarker level. The data thus obtained related to the level change of the biomarker set is useful information for monitoring the change of the development possibility of schizophrenia. More specifically, based on the level variation of the biomarker set, determination whether the development possibility is increased, decreased, or not changed during the period from the former examination to the present examination can be made. If this evaluation is carried out in parallel with the treatment of schizophrenia, therapeutic effect can be confirmed, and a foreboding sign of recurrence of schizophrenia can be grasped. As a result of this, more suitable treatment policy can be decided. Accordingly, the present invention can make marked contribution to the maximization of therapeutic effect and the improvement of the QOL (quality of life) of the patient.

### 3. Schizophrenia Examination Kit

The present invention further provides a kit for examining development possibility of schizophrenia. The kit of the present invention includes reagents for detecting the protein molecules composing the schizophrenia marker set. A reagent is provided for each biomarker (protein molecule). The reagents are composed of substances showing specific binding properties to the corresponding biomarkers (hereinafter referred to as “binding molecule”). For example, a reagent for GART is the substance having specific binding properties to GART. Examples of the binding molecule include antibodies that specifically recognize biomarkers, nucleic acid aptamers, and peptide aptamers. The binding molecules are not particularly limited as to their kind and origin, as long as they have specific binding properties to the biomarkers to be used. In addition, when an antibody is used, it may be a polyclonal antibody, an oligoclonal antibody (a mixture of various antibodies), or a monoclonal antibody. The polyclonal antibody or oligoclonal antibody may be an IgG fraction derived from antiserum obtained by animal immunization, or an affinity-purified antibody by an antigen. The anti-biomarker antibody may be a fragment of an antibody such as Fab, Fab′, F(ab′)₂, scFv, or dsFv antibody.

The binding molecule may be prepared in a common procedure. If a commercial product is available, the commercial product may be used. For example, an antibody may be prepared by an immunological method, a phage display method, or a ribosome display method. The preparation of a polyclonal antibody by an immunological method may be carried out by the following procedure. An antigen (biomarker or its portion) is prepared, and an animal such as a rabbit is immunized using the antigen. An antigen is obtained by purification of a living body sample. Alternatively, a recombinant antigen may be used. The recombinant antigen can be prepared by, for example, a gene (or a portion of the gene) coding a biomarker is introduced into an appropriate host using a vector, and expressed in the recombinant cell thus obtained.

In order to enhance the immunological induction action, an antigen bound with a carrier protein may be used. Examples of the carrier protein include KLH (Keyhole Limpet Hemocyanin), BSA (Bovine Serum Albumin), and OVA (Ovalbumin). Binding between the antigen and the carrier protein can use, for example a carbodiimide method, a glutaraldehyde method, a diazo condensation method, or an MBS (maleimide benzoyloxy succinimide) method. On the other hand, an antigen prepared by expressing a biomarker molecule (or its portion) as a fused protein with GST, β-galactosidase, maltose-bound protein, or histidine (His) tag can be used. The fused protein can be purified simply by a general-purpose method.

As necessary, immunization is repeated, the blood is collected after the antibody titer is thoroughly increased, and the blood serum is obtained by, for example, centrifugation treatment. The antiserum thus obtained is subjected to affinity purification, and thus obtaining a polyclonal antibody.

On the other hand, the monoclonal antibody may be prepared by the following procedure. Firstly, immunization operation is carried out by the above-described procedure. As necessary, immunization is repeated, and antibody-producing cells are extracted from the immunized animal after the antibody titer is thoroughly increased. Secondly, the antibody-producing cells thus obtained and myeloma cells are fused to obtain hybridoma. Subsequently, the hybridoma is cloned from a single cell, and then a clone producing an antibody having high specificity to the target protein is selected. The culture solution of the selected clone is purified to obtain the desired antibody. Alternatively, the hybridoma is proliferated to the desired number or more, and then the cells are transplanted into the abdominal cavity of an animal (for example, mouse), and proliferated in the ascites fluid, and the ascites fluid is purified to obtain the desired antibody. Affinity chromatography using, for example, protein G, protein A is suitable for the purification of the above-described culture solution or ascites fluid. Alternatively, affinity chromatography including an immobilized antigen may be used. Other method such as ion exchange chromatography, gel filtration chromatography, ammonium sulfate fractionation, or centrifugation may be used. These methods may be used alone or in combination.

Providing that the specific binding properties to a biomarker are maintained, the antibody thus obtained may be subjected to various modifications. The modified antibody may be used as the reagent in the present invention.

When the specific binding molecule is a labeling antibody, the amount of bound antibody can be directly detected using the amount of labeling as the index. Accordingly, the test method is more simplified. On the other hand, there are problems that an antibody bound with a labeling agent must be prepared, and that the detection sensitivity is commonly low. Therefore, an indirect detection method such as a method using a secondary antibody bound to a labeling agent, or a method using a polymer bound to a secondary antibody and a labeling agent is preferred. The secondary antibody herein is an antibody having specific binding properties to the antibody specific to the biomarker to be used. For example, when an antibody specific to the biomarker is prepared as a rabbit antibody, an anti-rabbit IgG antibody may be used as a secondary antibody. Labeled secondary antibodies usable for various types of antibodies such as rabbit, goat, and mouse antibodies are commercially available (for example, Funakoshi Co., Ltd. and Cosmo Bio Co., Ltd.), and suitable one may be appropriately selected according to the reagent in the present invention.

Examples of the labeling agent include enzymes such as peroxidase, microperoxidase, horseradish peroxidase (HRP), alkaline phosphatase, β-D-galactosidase, glucose oxidase, and glucose-6-phosphate dehydrogenase; fluorescent substances such as fluorescein isothiocyanate (FITC), tetramethyl rhodamine isothiocyanate (TRITC), and europium; chemiluminescence substances such as luminol, isoluminol, and acridinium derivatives; coenzymes such as NAD; biotin; and radioactive materials such as ¹³¹I and ¹²⁵I.

In one embodiment, the reagent in the present invention is solid-phased according to the intended use. The insoluble support used for solidification is not particularly limited. For example, an insoluble support made of water-insoluble substance such as a resin including a polystyrene resin, a polycarbonate resin, a silicon resin, and a nylon resin, or glass. The insoluble support carries an antibody by physical adsorption or chemical adsorption.

The kit of the present invention usually includes an instruction manual. The kit may include other reagents (for example, a buffer, a blocking reagent, an enzymatic substrate, and a color-producing reagent) used when carrying out the examination method and/or an apparatus or instrument (for example, a container, a reaction apparatus, an absorbance meter, and a fluorescence reader). In addition, the kit preferably includes, as the standard samples, the protein molecules or their fragments composing the biomarker set.

### Examples

The preceding investigation identified 22 proteins that were found to be related with schizophrenia (Patent Literature 1). With the aim of specifying the combination of biomarkers, which allow highly accurate discrimination, the following study was carried out.

1. Expression analysis by Western blot test

(1) Method

For the subjects composed of thirty patients with schizophrenia (15 men: 43.7±12.6 years of age, 15 women: 43.1±8.9 years of age) and thirty healthy subjects (15 men: 44.4±12.9 years of age, 15 women: 43.2±9.6 years of age), the amount of expression of 22 identified proteins was reconfirmed by the Western blot test method.

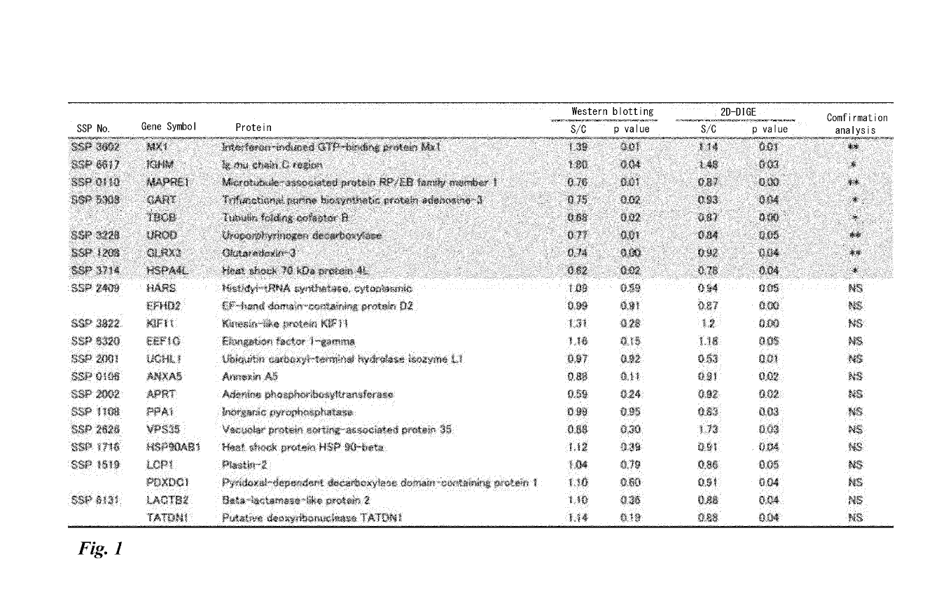

(2) Result Of the 22 proteins, eight proteins showed a significant difference in the expression amount (Student's t-test, p<0.05). Two proteins (MX1 and IGHM) showed increased expression, and six proteins (MAPRE1, TBCB, GLRX3, UROD, HSPA4L, and GART) showed decreased expression in the patient group with schizophrenia (FIG. 1).

2. Making of Disease Discriminant Model Using Statistical Method

The difference in the average of the amount of protein expression in the patient group with schizophrenia and the healthy subject group was compared using statistical hypothesis testing. As a result of this, eight proteins showed a significant difference (Student's t-test, p<0.05) (same as the above-described result). Of the eight proteins, the protein useful for the discrimination of the patient group with schizophrenia and the healthy subject group were identified, and the statistical model that can highly accurately discriminate the disease (logistic model) was made. The logistic model is a statistical model for discriminating the binary response represented by the following formula (formula (1)) (herein, the patient with schizophrenia and healthy subject).

$$\begin{matrix}[ Formula 2 ] & \\ p = \frac{exp ( {β}_{0} + {β}_{1} Protein 1 + … + {β}_{k} Protein k )}{1 + exp ( {β}_{0} + {β}_{1} Protein 1 + … + {β}_{k} Protein k )} & ( 1 )\end{matrix}$$

Wherein, p represents the probability of schizophrenia, and protein 1 to protein k represent the expression amount of the proteins used in discrimination. β₀ is the intercept and β₁ to βₖ are the parameters expressing the level of influence of the amounts of protein expression on the probability of schizophrenia and are presumed from the protein expression data. When β is positive (negative), the higher the amount of protein expression, the higher (lower) the probability of schizophrenia. In order to make a highly accurate model for discriminating the disease, the optimum combination must be selected from all the 255 combinations of proteins (=₈C₁+₈C₂+₈C₃+₈C₄+₈C₅+₈C₆+₈C₇+₈C₈). Using the statistical method below, two combinations of proteins (discriminant models) suitable for discrimination of the disease were identified.

<Discriminant Model 1>

For the 255 protein combinations, the predicting errors based on the bias corrected area under the curve (AUC), and leave one out cross validation (LOOCV) were calculated. The closer the corrected AUC to 1.0, the smaller the predicting error based on LOOCV, the higher the discrimination accuracy of the combination. These indices are evaluation indices recommended in statistical textbooks and literatures. FIG. 2 shows the top 30 combinations of proteins having a high corrected AUC. The table in FIG. 2 indicates that the model using GART, UROD, MX1, GLRX3, MAPRE1, and TBCB (hereinafter referred to as discriminant model 1) achieved the highest discrimination accuracy (corrected AUC: 0.877, predicting error: 0.164). The logistic model of the discriminant model 1 is as follows (formula (2)).

$$\begin{matrix}[ Formula 3 ] & \\ p = \frac{exp ( \begin{matrix}\begin{matrix}11.54 - 4.64 GART - 2.67 UROD + \\ 2.04 MX 1 - 3.14 GLRX 3 -\end{matrix} \\ 1.65 MARPRE 1 - 2.01 TBCB\end{matrix} )}{1 + exp ( \begin{matrix}\begin{matrix}11.54 - 4.64 GART - 2.67 UROD + \\ 2.04 MX 1 - 3.14 GLRX 3 -\end{matrix} \\ 1.65 MARPRE 1 - 2.01 TBCB\end{matrix} )} & ( 2 )\end{matrix}$$

The formula (2) indicates that, the higher the MX1, the higher the probability of schizophrenia. For other proteins, the higher the value, the lower the probability of schizophrenia.

<Discriminant Model 2>

Occam's razor is a statistical concept: “when an event is explained, the number of assumptions is preferably fewer”. Specifically, when a dependent variant (presence or absence of schizophrenia) is explained using a statistical model based on the measurement data of a plurality of explanation variables (proteins), conformity to the “measurement data” is improved as the number of explaining variables included in the statistical model is increased to complicate the model. However, this statistical model excessively conforms to “the measurement data (past data)”, and conformity to the new measured data decreases. More specifically, the statistical model overfits to the existing data. In order to avoid this problem, the number of explaining variables included in the statistical model is preferably fewer. Based on this concept, when the discrimination accuracy is on a similar level, the number of proteins used for the discrimination is preferably fewer. From the table in FIG. 2, the model using GART, UROD, MX1, and GLRX3 (hereinafter referred to as discriminant model 2) is suitable for the idea (corrected AUC: 0.860, predicting error: 0.178). The logistic model of the discriminant model 2 (formula (3)) is as follows.

$$\begin{matrix}[ Formula 4 ] & \\ p = \frac{exp ( \begin{matrix}6.44 - 3.82 GART - 2.18 UROD + \\ 2.48 MX 1 - 2.84 GLRX 3\end{matrix} )}{1 + exp ( \begin{matrix}6.44 - 3.82 GART - 2.18 UROD + \\ 2.48 MX 1 - 2.84 GLRX 3\end{matrix} )} & ( 3 )\end{matrix}$$

The positive and negative of the parameter are the same as those in the discriminant model 1, and the influences of the proteins on the probability of schizophrenia are the same as those in the discriminant models 1 and 2.

<Comparison Between Discriminant Models 1 and 2>

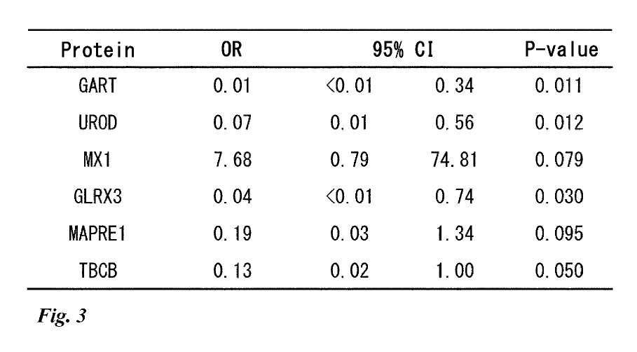

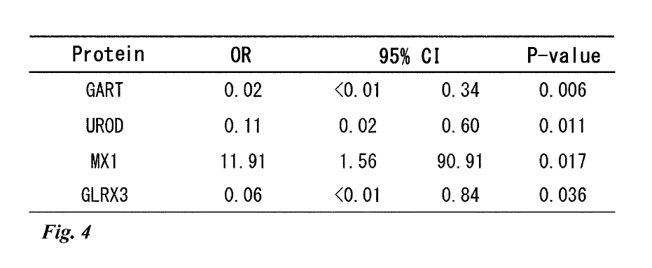

The odds ratios of the proteins contained in the discriminant models 1 and 2 are shown in FIGS. 3 and 4, respectively. The influence of the protein contained in these models on the prevalence rate of schizophrenia is statistically significant (p<0.05) or limitedly significant (p<0.10), and is suggested to be a protein suitable for discrimination. In addition, the combination of proteins suitable for discrimination was selected using stepwise forward selection method, which is a statistical method. As a result of this, GART, UROD, MX1, and GLRX3 were selected, so that usefulness of the discriminant model 2 was supported by the different statistical method. Furthermore, the table in FIG. 2 shows that the number contained in the model is higher in the order of GART, UROD, MX1, GLRX3, MAPRE1, TBCB, IGHM, and HSPA4L.

The above results suggest the followings.
The accuracy of discrimination of schizophrenia by the discriminant models 1 and 2 are almost equal statistically.
GART, UROD, MX1, and GLRX3 contained in almost all the models in FIG. 2 are particularly important for the discrimination of schizophrenia.
MARPRE1 and TBCB contained in the discriminant model 1 are also highly useful for discrimination.

<Determination of Schizophrenia Using Discriminant Models>

When determining whether the patient has schizophrenia or not using the discriminant model 1 or 2, the prevalence rate of schizophrenia (p in the formula (2) or (3)) is calculated. For example, when the discriminant model 1 is used, GART, UROD, MX1, GLRX3, MAPRE1, and TBCB in the patient are measured, and the values are assigned to the formula (2), thereby determining the prevalence rate of schizophrenia.

3. Validation of Effectiveness of Biomarker

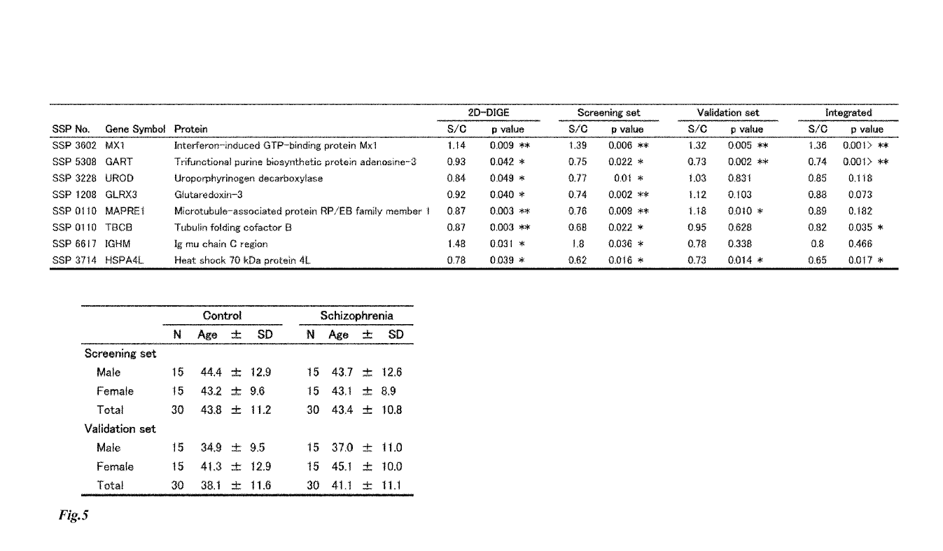

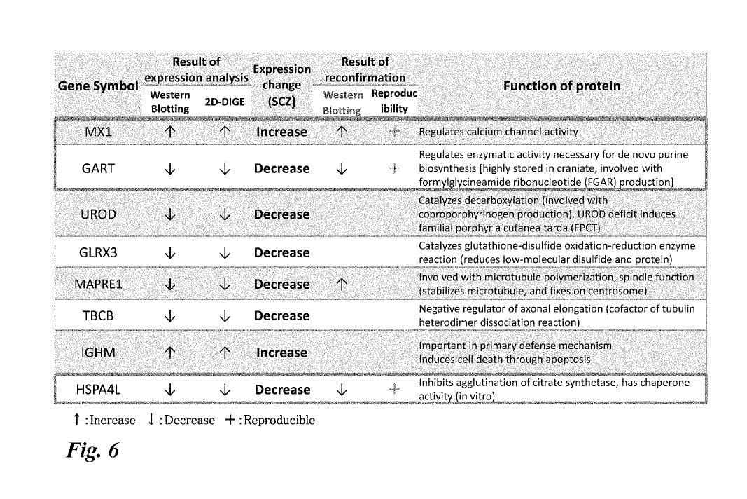

Effectiveness of the selected eight biomarkers was validated using other clinical specimens. The results of validation by Western blot test are shown in FIGS. 5 and 6. For MX1, GART, and HSPA4L, the screening data (the data of 30 specimens used in the above-described 1.) and the validation data (the data of other 30 specimens) changed in the same direction (statistically significant), and showed reproducibility.

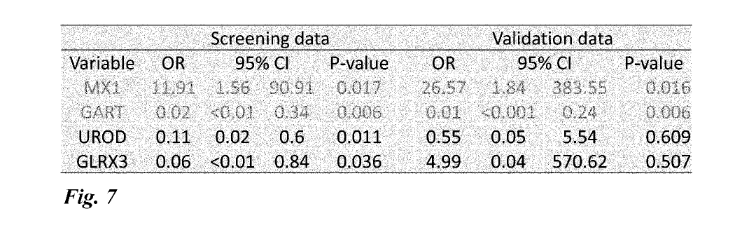

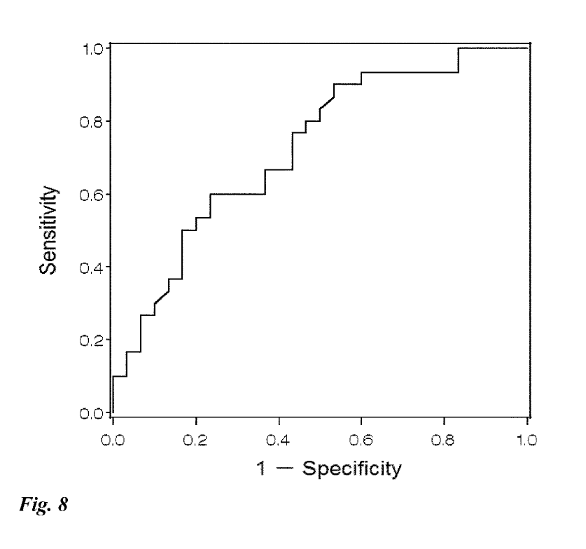

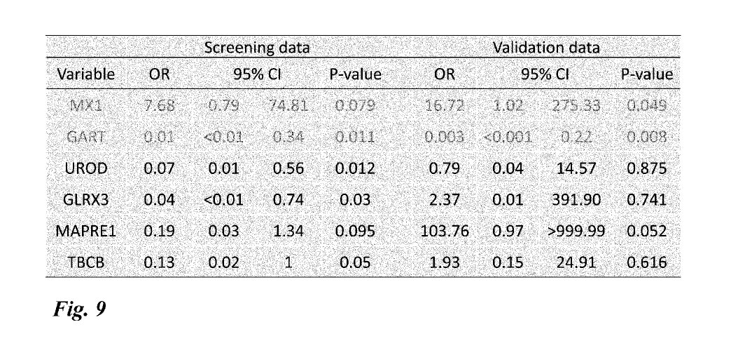

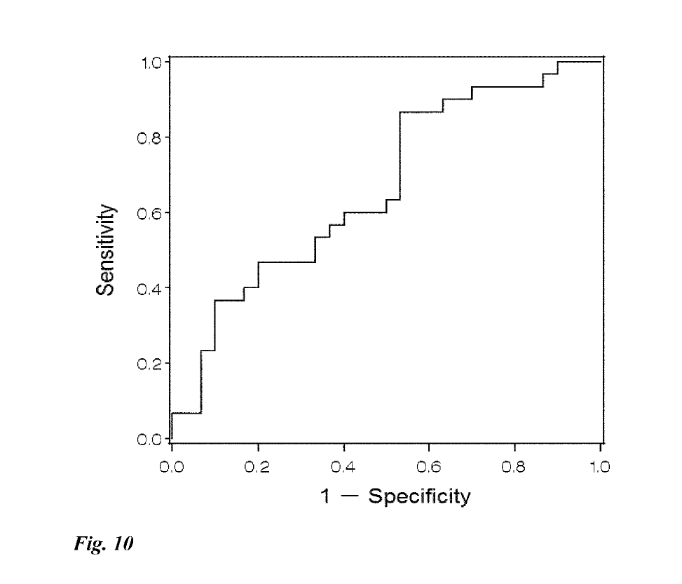

Effectiveness of the four biomarkers (MX1, GART, UROD, and GLRX3) was validated using the validation data set; significance was reproduced for MX1 and GART (FIGS. 7 and 8). In addition, also when effectiveness of the six biomarkers (MX1, GART, UROD, GLRX3, MAPRE1, and TBCB) was validated using the validation data set, significance was reproduced for MX1 and GART (FIGS. 9 and 10). The four-variable model showed a higher predicting accuracy.

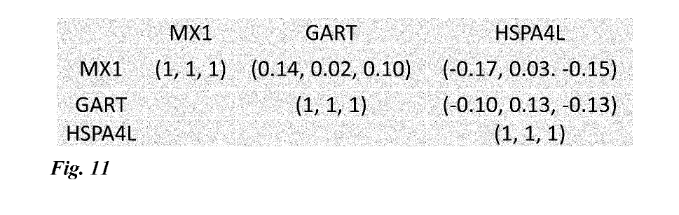

In consideration that MX1, GART, and HSPA4L showed reproducibility, the correlation between these three molecules was studied by multivariate analysis. The result is shown in FIG. 11. The values in parentheses are, from left to right, correlation coefficient in screening data, correlation coefficient in validation data, and correlation coefficient in the integration of both data. There is no correlation between the three molecules. The cause that HSPA4L showed no significance in the multivariate analysis is not likely due to the influence of multi-collinearity.

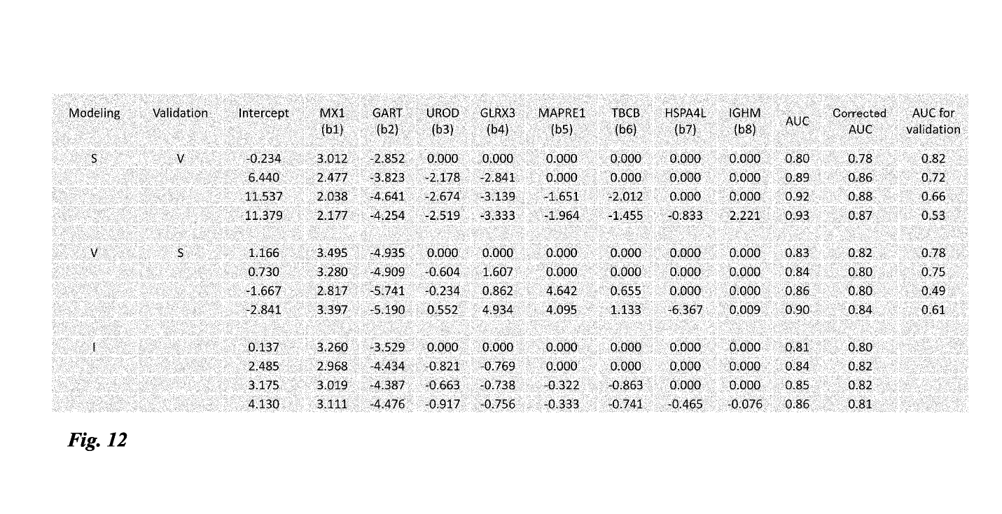

On the other hand, a plurality of predicting models including different types and numbers of biomarkers were constructed using eight biomarkers (MX1, GART, UROD, GLRX3, MAPRE1, TBCB, HSPA4L, IGHM), with focusing attention on MX1 and GART which reproduced significance, and further validation was carried out. The validation results are shown in FIG. 12. Except for some predicting models, high reproducibility is shown. From the viewpoints of predicting accuracy and reproducibility, the two variable models, MX1 and GART are the most advantageous. The predicting models are represented in the same manner as the above-described formula (1). For example, the discriminant model in the top row in FIG. 12 is represented by: p=exp(−0.234+3.012MX1−2.852GART)/(1+exp(−0.234+3.012MX1−2.852GART).

The above results suggest the followings.
MX1 and GART have good reproducibility, and thus are advantageous biomarkers.
HSPA4L also has relatively good reproducibility, and is highly useful.
Discrimination using MX1 and GART gives the most accurate result.

### INDUSTRIAL APPLICABILITY

The examination method of the present invention allows highly accurate discrimination of schizophrenia. The examination method of the present invention is useful as a means for determining whether schizophrenia is developed or not. It is also useful as a means for grasping the future development possibility. Early detection and treatment using the examination method of the present invention are considered effective for the prevention of increase in obstinacy and seriousness (progress of disease), and recurrence of schizophrenia.

The present invention will not be limited to the description of the embodiments and examples of the present invention. Various modifications readily made by those skilled in the art are also included in the present invention, without departing from the scope of claims. The entire contents of the articles, unexamined patent publications, patent applications, and the like specified herein are hereby incorporated herein by reference.

### SEQUENCE LIST

1. A schizophrenia marker set comprising a combination of two or more protein molecules selected from the group consisting of trifunctional purine biosynthetic protein adenosine-3, uroporphyrinogen decarboxylase, interferon-induced GTP-binding protein Mx1, glutaredoxin-3, microtubule-associated protein RP/EB family member 1, tubulin folding cofactor B, immunoglobulin mu chain C region, and heat shock 70 kDa protein 4L.

2. The schizophrenia marker set of claim 1, which comprises at least trifunctional purine biosynthetic protein adenosine-3, uroporphyrinogen decarboxylase, interferon-induced GTP-binding protein Mx1, and glutaredoxin-3.

3. The schizophrenia marker set of claim 2, which further comprises microtubule-associated protein RP/EB family member 1 and/or tubulin folding cofactor B.

4. The schizophrenia marker set of claim 1, which is a combination of trifunctional purine biosynthetic protein adenosine-3, uroporphyrinogen decarboxylase, interferon-induced GTP-binding protein Mx1, glutaredoxin-3, microtubule-associated protein RP/EB family member 1, and tubulin folding cofactor B.

5. The schizophrenia marker set of claim 1, which is a combination of trifunctional purine biosynthetic protein adenosine-3, uroporphyrinogen decarboxylase, interferon-induced GTP-binding protein Mx1, and glutaredoxin-3.

6. The schizophrenia marker set of claim 1, which comprises at least trifunctional purine biosynthetic protein adenosine-3 and interferon-induced GTP-binding protein Mx1.

7. The schizophrenia marker set of claim 1, which is a combination of trifunctional purine biosynthetic protein adenosine-3 and interferon-induced GTP-binding protein Mx1.

8. The schizophrenia marker set of claim 1, which comprises one or more protein molecules selected from the group consisting of trifunctional purine biosynthetic protein adenosine-3, interferon-induced GTP-binding protein Mx1, and heat shock 70 kDa protein 4L.

9. An examination method for schizophrenia using the level of the schizophrenia marker set of claim 1 in a specimen as an indication.

10. The examination method for schizophrenia of claim 9, comprising the following steps (1) to (3):
(1) a step of preparing a specimen derived from the subject;
(2) a step of detecting the protein molecules composing the marker set in the specimen; and
(3) a step of determining the present or future development possibility of schizophrenia based on the detection result.

11. The examination method for schizophrenia of claim 10, wherein a lower detected value shows a higher development possibility of schizophrenia for trifunctional purine biosynthetic protein adenosine-3, uroporphyrinogen decarboxylase, glutaredoxin-3, microtubule-associated protein RP/EB family member 1, tubulin folding cofactor B, and heat shock 70 kDa protein 4L, and a higher detected value shows a higher development possibility of schizophrenia for interferon-induced GTP-binding protein Mx1 and immunoglobulin mu chain C region.

12. The examination method for schizophrenia of claim 10, wherein the determination of step (3) is carried out using a logistic model.

13. The examination method for schizophrenia of claim 9, wherein the specimen is blood, blood plasma, blood serum, oral mucosa, nasal mucosa, skin, blood cells, or lymphoblastoid cells prepared by immortalizing the blood lymphocytes collected from the subject.

14. A kit for examining schizophrenia comprising a reagent for detecting each of the protein molecules composing the schizophrenia marker set of claim 1, and an instruction manual.

15. The kit for examining schizophrenia of claim 14, wherein each of the reagents is an antibody to the target protein molecule.


SEQUENCE LISTING (8 sequences)

SEQ ID NO: 1 - protein, 1010 aa, Homo sapiens
MAARVLIIGSGGREHTLAWKLAQSHHVKQVLVAPGNAGTACSEKISNTAISISDHTALAQFCKEKKIEFVVVGPEAPLAAGIVGNLRSAGVQCFGPTAEAAQLESSKRFAKEFMDRHGIPTAQWKAFTKPEEACSFILSADFPALVVKASGLAAGKGVIVAKSKEEACKAVQEIMQEKAFGAAGETIVIEELLDGEEVSCLCFTDGKTVAPMPPAQDHKRLLEGDGGPNTGGMGAYCPAPQVSNDLLLKIKDTVLQRTVDGMQQEGTPYTGILYAGIMLTKNGPKVLEFNCRFGDPECQVILPLLKSDLYEVIQSTLDGLLCTSLPVWLENHTALTVVMASKGYPGDYTKGVEITGFPEAQALGLEVFHAGTALKNGKVVTHGGRVLAVTAIRENLISALEEAKKGLAAIKFEGAIYRKDVGFRAIAFLQQPRSLTYKESGVDIAAGNMLVKKIQPLAKATSRSGCKVDLGGFAGLFDLKAAGFKDPLLASGTDGVGTKLKIAQLCNKHDTIGQDLVAMCVNDILAQGAEPLFFLDYFSCGKLDLSVTEAVVAGIAKACGKAGCALLGGETAEMPDMYPPGEYDLAGFAVGAMERDQKLPHLERITEGDVVVGIASSGLHSNGFSLVRKIVAKSSLQYSSPAPDGCGDQTLGDLLLTPTRIYSHSLLPVLRSGHVKAFAHITGGGLLENIPRVLPEKLGVDLDAQTWRIPRVFSWLQQEGHLSEEEMARTFNCGVGAVLVVSKEQTEQILRDIQQHKEEAWVIGSVVARAEGSPRVKVKNLIESMQINGSVLKNGSLTNHFSFEKKKARVAVLISGTGSNLQALIDSTREPNSSAQIDIVISNKAAVAGLDKAERAGIPTRVINHKLYKNRVEFDSAIDLVLEEFSIDIVCLAGFMRILSGPFVQKWNGKMLNIHPSLLPSFKGSNAHEQALETGVTVTGCTVHFVAEDVDAGQIILQEAVPVKRGDTVATLSERVKLAEHKIFPAALQLVASGTVQLGENGKICWVKEE

SEQ ID NO: 2 - protein, 367 aa, Homo sapiens
MEANGLGPQGFPELKNDTFLRAAWGEETDYTPVWCMRQAGRYLPEFRETRAAQDFFSTCRSPEACCELTLQPLRRFPLDAAIIFSDILVVPQALGMEVTMVPGKGPSFPEPLREEQDLERLRDPEVVASELGYVFQAITLTRQRLAGRVPLIGFAGAPWTLMTYMVEGGGSSTMAQAKRWLYQRPQASHQLLRILTDALVPYLVGQVVAGAQALQLFESHAGHLGPQLFNKFALPYIRDVAKQVKARLREAGLAPVPMIIFAKDGHFALEELAQAGYEVVGLDWTVAPKKARECVGKTVTLQGNLDPCALYASEEEIGQLVKQMLDDFGPHRYIANLGHGLYPDMDPEHVGAFVDAVHKHSRLLRQN

SEQ ID NO: 3 - protein, 662 aa, Homo sapiens
MVVSEVDIAKADPAAASHPLLLNGDATVAQKNPGSVAENNLCSQYEEKVRPCIDLIDSLRALGVEQDLALPAIAVIGDQSSGKSSVLEALSGVALPRGSGIVTRCPLVLKLKKLVNEDKWRGKVSYQDYEIEISDASEVEKEINKAQNAIAGEGMGISHELITLEISSRDVPDLTLIDLPGITRVAVGNQPADIGYKIKTLIKKYIQRQETISLVVVPSNVDIATTEALSMAQEVDPEGDRTIGILTKPDLVDKGTEDKVVDVVRNLVFHLKKGYMIVKCRGQQEIQDQLSLSEALQREKIFFENHPYFRDLLEEGKATVPCLAEKLTSELITHICKSLPLLENQIKETHQRITEELQKYGVDIPEDENEKMFFLIDKVNAFNQDITALMQGEETVGEEDIRLFTRLRHEFHKWSTIIENNFQEGHKILSRKIQKFENQYRGRELPGFVNYRTFETIVKQQIKALEEPAVDMLHTVTDMVRLAFTDVSIKNFEEFFNLHRTAKSKIEDIRAEQEREGEKLIRLHFQMEQIVYCQDQVYRGALQKVREKELEEEKKKKSWDFGAFQSSSATDSSMEEIFQHLMAYHQEASKRISSHIPLIIQFFMLQTYGQQLQKAMLQLLQDKDTYSWLLKERSDTSDKRKFLKERLARLTQARRRLAQFPG

SEQ ID NO: 4 - protein, 335 aa, Homo sapiens
MAAGAAEAAVAAVEEVGSAGQFEELLRLKAKSLLVVHFWAPWAPQCAQMNEVMAELAKELPQVSFVKLEAEGVPEVSEKYEISSVPTFLFFKNSQKIDRLDGAHAPELTKKVQRHASSGSFLPSANEHLKEDLNLRLKKLTHAAPCMLFMKGTPQEPRCGFSKQMVEILHKHNIQFSSFDIFSDEEVRQGLKAYSSWPTYPQLYVSGELIGGLDIIKELEASEELDTICPKAPKLEERLKVLTNKASVMLFMKGNKQEAKCGFSKQILEILNSTGVEYETFDILEDEEVRQGLKAYSNWPTYPQLYVKGELVGGLDIVKELKENGELLPILRGEN

SEQ ID NO: 5 - protein, 281 aa, Homo sapiens
MAVNVYSTSVTSENLSRHDMLAWVNDSLHLNYTKIEQLCSGAAYCQFMDMLFPGCVHLRKVKFQAKLEHEYIHNFKVLQAAFKKMGVDKIIPVEKLVKGKFQDNFEFIQWFKKFFDANYDGKDYNPLLARQGQDVAPPPNPGDQIFNKSKKLIGTAVPQRTSPTGPKNMQTSGRLSNVAPPCILRKNPPSARNGGHETDAQILELNQQLVDLKLTVDGLEKERDFYFSKLRDIELICQEHESENSPVISGIIGILYATEEGFAPPEDDEIEEHQQEDQDEY

SEQ ID NO: 6 - protein, 244 aa, Homo sapiens
MEVTGVSAPTVTVFISSSLNTFRSEKRYSRSLTIAEFKCKLELLVGSPASCMELELYGVDDKFYSKLDQEDALLGSYPVDDGCRIHVIDHSGARLGEYEDVSRVEKYTISQEAYDQRQDTVRSFLKRSKLGRYNEEERAQQEAEAAQRLAEEKAQASSIPVGSRCEVRAAGQSPRRGTVMYVGLTDFKPGYWIGVRYDEPLGKNDGSVNGKRYFECQAKYGAFVKPAVVTVGDFPEEDYGLDEI

SEQ ID NO: 7 - protein, 453 aa, Homo sapiens [sequence omitted]

SEQ ID NO: 8 - protein, 839 aa, Homo sapiens
MSVVGIDLGFLNCYIAVARSGGIETIANEYSDRCTPACISLGSRTRAIGNAAKSQIVTNVRNTIHGFKKLHGRSFDDPIVQTERIRLPYELQKMPNGSAGVKVRYLEEERPFAIEQVTGMLLAKLKETSENALKKPVADCVISIPSFFTDAERRSVMAAAQVAGLNCLRLMNETTAVALAYGIYKQDLPPLDEKPRNVVFIDMGHSAYQVLVCAFNKGKLKVLATTFDPYLGGRNFDEALVDYFCDEFKTKYKINVKENSRALLRLYQECEKLKKLMSANASDLPLNIECFMNDLDVSSKMNRAQFEQLCASLLARVEPPLKAVMEQANLQREDISSIEIVGGATRIPAVKEQITKFFLKDISTTLNADEAVARGCALQCAILSPAFKVREFSITDLVPYSITLRWKTSFEDGSGECEVFCKNHPAPFSKVITFHKKEPFELEAFYTNLHEVPYPDARIGSFTIQNVFPQSDGDSSKVKVKVRVNIHGIFSVASASVIEKQNLEGDHSDAPMETETSFKNENKDNMDKMQVDQEEGHQKCHAEHTPEEEIDHTGAKTKSAVSDKQDRLNQTLKKGKVKSIDLPIQSSLCRQLGQDLLNSYIENEGKMIMQDKLEKERNDAKNAVEEYVYDFRDRLGTVYEKFITPEDLSKLSAVLEDTENWLYEDGEDQPKQVYVDKLQELKKYGQPIQMKYMEHEERPKALNDLGKKIQLVMKVIEAYRNKDERYDHLDPTEMEKVEKCISDAMSWLNSKMNAQNKLSLTQDPVVKVSEIVAKSKELDNFCNPIIYKPKPKAEVPEDKPKANSEHNGPMDGQSGTETKSDSTKDSSQHTKSSGEMEVD